(19) United States
(12) Patent Application Publication
(10) Pub. No.: US 2006/0083790 A1
(43) Pub. Date: Apr. 20, 2006

(54) Combination of an ibat inhibitor and a metal salt for the treatment of diarrhoea

(76) Inventors: Eva-Karin Anderberg, Molndal (SE); Erik Soderlind, Molndal (SE)

Correspondence Address: MORGAN LEWIS & BOCKIUS LLP, 1111 PENNSYLVANIA AVENUE NW, WASHINGTON, DC, 20004, US

(21) Appl. No.: 10/520,939

(22) PCT Filed: Jul. 9, 2003
(86) PCT No.: PCT/GB03/02978

§ 371 Date: Jan. 12, 2005

(30) Foreign Application Priority Data
Jul. 13, 2002 (GB) 0216321.0

(51) Int. Cl. A61K 31/554; A61K 33/42

(52) U.S. Cl. 424/602; 514/211.07

(57) ABSTRACT

A combination comprising an IBAT inhibitor, or a pharmaceutically acceptable salt, solvate of such a salt or a prodrug thereof, and a metal salt, wherein the metal salt is formulated to release in the terminal ileum, caecum and/or the colon, is described. Compositions containing this combination and uses of the combination are also described.

The present invention relates to combination treatments comprising a metal salt and compounds that possess ileal bile acid transport (IBAT) inhibitory activity wherein the metal salt is formulated to release in the terminal ileum, caecum and/or the colon. These combination treatments are useful in preventing diarrhoea that would result from excess bile acids in the intestine following administration of an effective amount an IBAT inhibitor, or a pharmaceutically acceptable salt, solvate, solvate of such a salt or a prodrug thereof, to a warm-blooded animal, such as man. The invention also relates to pharmaceutical compositions containing these combinations and to their use in the manufacture of medicaments. These combinations have value in the treatment of disease states associated with hyperlipidaemic conditions.

It is well-known that hyperlipidaemic conditions associated with elevated concentrations of total cholesterol and LDL cholesterol are major risk factors for cardiovascular atherosclerotic disease (Circulation 1999, 100, 1930-1938 and Circulation, 1999, 100, 1134-46). To reduce the risk and the total mortality due to cardiovascular disease, the reduction of plasma lipids, particularly LDL cholesterol, is now recognized as an important therapeutic goal (N Engl J. Med. 1995; 332:5, 12-21).

Interfering with the circulation of bile acids within the lumen of the intestinal tracts has also been found to reduce the level of cholesterol. Bile acids are synthesized in the liver from cholesterol and secreted into the bile. They are actively recycled (>95%) from the small intestine back to the liver. Previous established therapies have involved, for example, treatment with bile acid binders, such as resins. Frequently used bile acid binders are for instance cholestyramine and cholestipol.

Another proposed therapy (Current Opinion on Lipidology, 1999, 10, 269-74) involves the treatment with substances with an IBAT inhibitory effect. Theoretically, IBAT inhibitors should have similar therapeutic effect as the resins but they might also be expected to have attractive advantages. First, it should be possible to administer IBAT inhibitors as tablets at the same dose intervals as statins. Second, a direct inhibition of the transport of bile acids across the ileum should be advantageous in situations when IBAT is upregulated. However, available data on the effects of IBAT inhibitors is limited. Several IBAT agents have previously been shown to promote the faecal excretion of bile acids and to reduce plasma cholesterol. The proposed mechanism for the hypolipidaemic action of these compounds is by an induced number of hepatic LDL receptors due to the increased consumption of hepatic cholesterol caused by a compensatory increased bile acid synthesis (Arterioscler Thromb Vasc Biol. 1998; 18: 1304-11).

However, bile acids that are not recycled in the intestines induce irritation of the intestinal luminal surfaces, at least at higher concentrations. This is seen for example in chronic diarrhoea, and in post infectious diarrhoea with deficient uptake of bile acids, after continuous bile acid secretion following cholecystectomy and after resection of the distal ileum. In vivo dosing of IBAT compounds may give rise to these side effects either in certain patients or at high enough doses, i.e. irritation of the intestine would be induced, resulting in diarrhoea. The present invention ameliorates this problem.

Furthermore, if chronic diarrhoea was a side effect, then it is possible that these compounds would not be suitable for administering to patients at all (or at least at high enough doses to give a therapeutic effect), despite their efficacy. The present invention therefore provides the additional advantage that it opens up treatment with an IBAT inhibitor to a particular patient population where it might otherwise have not been possible to use these compounds.

Patients suffering from bile acid induced diarrhoea caused by intestinal bypass for example have previously been treated with large doses (2-4 g) of a calcium salt (Reference: Steinbach et al Eur. J. of Gastroenterology & Hepathology 1996, 8:559-562). A 2-4 g dose of a salt is too large for convenient dosing regimen, and patient compliance with this regime would be in doubt. This dose is also too large to make a single tablet made up of the IBAT inhibitor and the salt, which is one aspect of the present invention. A formulation which delivers the metal salt with a targeted release to the terminal ileum, caecum and/or the colon would allow a much lower dose of the salt to be used because there will be no loss of the metal salt due to absorption or binding to other components in the small intestine. Therefore it should be possible to formulate a convenient combination regimen, either a single combination tablet or otherwise.

In the literature IBAT inhibitors are often referred to by different names. It is to be understood that where IBAT inhibitors are referred to herein, this term also encompasses compounds known in the literature as:
i) ileal apical sodium co-dependent bile acid transporter (ASBT) inhibitors;
ii) bile acid transporter (BAT) inhibitors;
iii) ileal sodium/bile acid cotransporter system inhibitors;
iv) apical sodium-bile acid cotransporter inhibitors;
v) ileal sodium-dependent bile acid transport inhibitors;
vi) bile acid reabsorption (BARI's) inhibitors; and
vii) sodium bile acid transporter (SBAT) inhibitors;
   where they act by inhibition of IBAT.

Accordingly the present invention provides a combination which comprises an IBAT inhibitor, or a pharmaceutically acceptable salt, solvate, solvate of such a salt or a prodrug thereof, and a metal salt, wherein the metal salt is formulated to release in the terminal ileum, caecum and/or the colon.

The present inventors have found that there are at least two mechanisms behind the calcium induced bile acid binding. Firstly, bile acids may adsorb to calcium phosphate particles, and, secondly, unconjugated bile acids may form insoluble calcium salts of bile acids.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the benefit of the combination.

The combination of the present invention may either be in the form of a fixed combination with the IBAT inhibitor, in which case both the IBAT inhibitor and the metal salt are formulated to release in the terminal ileum, caecum and/or the colon, or a free combination wherein only the metal salt is formulated to release in the terminal ileum, caecum and/or the colon.

In one aspect, the metal salt is formulated to release in the terminal ileum. In a further aspect the metal salt is formulated to release in the caecum. In another aspect of the invention, the metal salt is formulated to release in the colon. In one aspect, the metal salt is formulated to release in the terminal ileum and the caceum. In a further aspect the metal salt is formulated to release in the caecum and the colon. In another aspect of the invention, the metal salt is formulated to release in the terminal ileum and the colon. In another aspect of the invention the metal salt is formulated to release in the terminal ileum, caecum and the colon.

In another aspect where the metal salt is formulated to release in a specified site, i.e. the terminal ileum, caecum and/or the colon, particularly greater than 50% of the metal salt is released here. More particularly this is greater than 70%. More particularly this is greater than 90%. More particularly this is greater than 95%. More particularly this is greater than 99%.

Suitable metals in the metal salt include any pharmaceutically acceptable multivalent metal ion. In one aspect of the invention these metals are calcium, aluminium, iron, copper, zinc, magnesium, manganese or tin salts. In another aspect of the invention these metals are Ca(II), Al(III), Fe(II), Fe(III), Cu(II), Zn(II), Mg(II), Mn(II) or Sn(II) salts. In a further aspect of the invention the metal in the metal salt is calcium. In another aspect the metal in the metal salt is Ca(II). The salt may be any suitable pharmaceutically acceptable salt. In one aspect the salt is acetate, ascorbate, carbonate, chloride, citrate, gluconate, lactate, nitrate, oxalate, phosphate or sulphate. Suitable metal salts include calcium phosphate, calcium lactate, calcium carbonate, calcium gluconate and calcium acetate, particularly calcium phosphate.

It is to be understood that the combination of the present invention includes the situation where there is one metal salt in the combination with the IBAT inhibitor. In addition the combination of the present invention includes the situation where there are one or more metal salts in the combination with the IBAT inhibitor. In this case the salts may be one or more different salts of the same metal, one or more of the same salt of different metals or one or more different salts of different metals.

Suitable compounds possessing IBAT inhibitory activity have been described, see for instance the compounds described in WO 93/16055, WO 94/18183, WO 94/18184, WO 96/05188, WO 96/08484, WO 96/16051, WO 97/33882, WO 98/38182, WO 98/40375, WO 99/35135, WO 99/64409, WO 99/64410, WO 00/01687, WO 00/38725, WO 00/38726, WO 00/38727, WO 00/38728, WO 00/38729, WO 00/47568, WO 00/61568, WO 01/66533, DE 19825804, and EP 864 582 and the contents of these patent applications are incorporated herein by reference. Particularly the named examples of these patent applications are incorporated herein by reference. More particularly claim 1 of these patent application are incorporated herein by reference.

Further suitable compounds possessing IBAT inhibitory activity have been described in WO 94/24087, WO 98/07749, WO 98/56757, WO 99/32478, WO 00/20392, WO 00/20393, WO 00/20410, WO 00/20437, WO 00/35889, WO 01/34570, WO 01/68096, WO 01/68637, WO 02/08211, JP 10072371, U.S. Pat. No. 5,070,103, EP 251 315, EP 417 725, EP 489 423, EP 549 967, EP 573 848, EP 624 593, EP 624 594, EP 624 595, EP 869 121 and EP 1 070 703. Particularly the named examples of these patent applications are incorporated herein by reference. More particularly claim 1 of these patent application are incorporated herein by reference.

Particular classes of IBAT inhibitors suitable for use in the present invention are benzothiepines, and the compounds described in the claims, particularly claim 1, of WO 00/01687, WO 96/08484 and WO 97/33882 are incorporated herein by reference. Other suitable classes of IBAT inhibitors are the 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity is (3R,5R)-3-butyl-3-ethyl-1,1-dioxido-5-phenyl-2,3,4,5-tetrahydro-1,4-benzothiazepin-8-yl β-D-glucopyranosiduronic acid (EP 864 582).

A further suitable compound possessing IBAT inhibitory activity is S-8921 (EP 597 107).

A further suitable IBAT inhibitor is the compound:

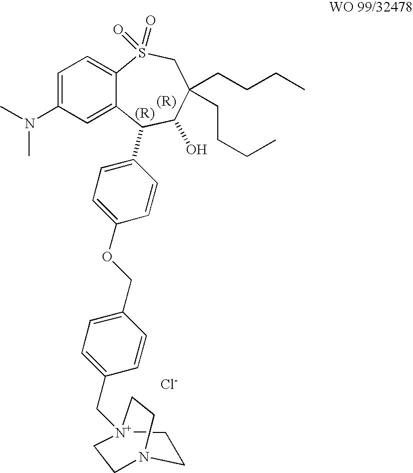

Other suitable IBAT inhibitors are those described in WO 01/66533. A particular compound of the invention is selected from any one of Example 1-39 of WO 01/66533, or a pharmaceutically acceptable salt, solvate, solvate of such a salt or a prodrug thereof, and the compounds of Examples 1-39 are incorporated herein by reference. Claims 1-6 of WO 01/66533 are also incorporated herein by reference.

Additional suitable IBAT inhibitors are those described in WO 02/50051. Additional suitable compounds possessing IBAT inhibitory activity have the following structure of formula (AI): 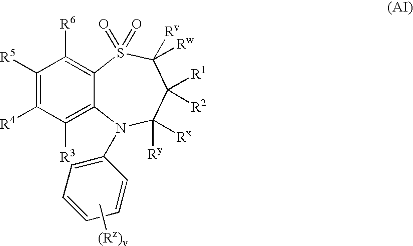
wherein:

R^{v} and R^{w} are independently selected from hydrogen or C₁₋₆alkyl;

R¹ and R² are independently selected from C₁₋₆alkyl;

R^{x} and R^{y} are independently selected from hydrogen or C₁₋₆alkyl, or one of R^{x} and R^{y} is hydrogen or C₁₋₆alkyl and the other is hydroxy or C₁₋₆alkoxy;

R^{z} is selected from halo, nitro, cyano, hydroxy, amino, carboxy, carbamoyl, mercapto, sulphamoyl, C₁₋₆alkyl, C₂ alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, C₁₋₆alkoxycarbonylamino, ureido, N′-(C₁₋₆alkyl)ureido, N-(C₁₋₆alkyl)ureido, N′,N′-(C₁₋₆alkyl)₂ureido, N′-(C₁₋₆alkyl)-N-(C₁₋₆alkyl)ureido, N′,N′-(C₁₋₆alkyl)₂-N-(C₁₋₆alkyl)ureido, N-(C₁₋₆alkyl)sulphamoyl and N,N-(C₁₋₆alkyl)₂sulphamoyl;

v is 0-5;

one of R⁴ and R⁵ is a group of formula (AIA):

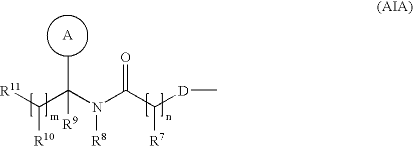

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;

D is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted by one or more substituents selected from R¹⁷;

R⁷ is hydrogen, C₁₋₄alkyl, carbocyclyl or heterocyclyl; wherein R⁷ is optionally substituted by one or more substituents selected from R¹⁸;

R⁸ is hydrogen or C₁₋₄alkyl;

R⁹ is hydrogen or C₁₋₄alkyl;

R¹⁰ is hydrogen, C₁₋₄alkyl, carbocyclyl or heterocyclyl; wherein R¹⁰ is optionally substituted by one or more substituents selected from R¹⁹;

R¹¹ is carboxy, sulpho, sulphino, phosphono, tetrazolyl, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (AIB): 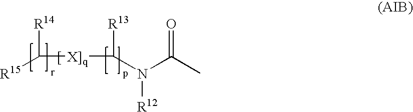
wherein:

X is —N(R^{q})—, —N(R^{q})C(O)—, —O—, and —S(O)ₐ—; wherein a is 0-2 and R^{q} is hydrogen or C₁₋₄alkyl;

R¹² is hydrogen or C₁₋₄alkyl;

R¹³ and R¹⁴ are independently selected from hydrogen, C₁₋₄alkyl, carbocyclyl, heterocyclyl or R²³; wherein said C₁₋₄alkyl, carbocyclyl or heterocyclyl may be independently optionally substituted by one or more substituents selected from R²⁰;

R¹⁵ is carboxy, sulpho, sulphino, phosphono, tetrazolyl, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆alkyl; or R¹⁵ is a group of formula (AIC): 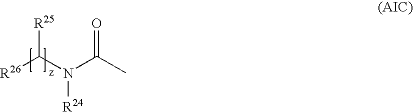
wherein:

R²⁴ is selected from hydrogen or C₁₋₄alkyl;

R² is selected from hydrogen, C₁₋₄alkyl, carbocyclyl, heterocyclyl or R²⁷; wherein said C₁₋₄alkyl, carbocyclyl or heterocyclyl may be independently optionally substituted by one or more substituents selected from R²⁸;

R²⁶ is selected from carboxy, sulpho, sulphino, phosphono, tetrazolyl, —P(O)(OR^{g})(OR^{h}), —P(O)(OH)(OR^{g}), —P(O)(OH)(R^{g}) or —P(O)(OR^{g})(R^{h}) wherein R^{g} and R^{h} are independently selected from C₁₋₆alkyl;

p is 1-3; wherein the values of R¹³ may be the same or different;

q is 0-1;

r is 0-3; wherein the values of R¹⁴ may be the same or different;

m is 0-2; wherein the values of R¹⁰ may be the same or different;

n is 1-3; wherein the values of R⁷ may be the same or different;

z is 0-3; wherein the values of R²⁵ may be the same or different;

R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;

R¹⁹, R²⁰, R²³, R²⁷ and R²⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹, R²⁰, R²³, R²⁷ and R²⁸ may be independently optionally substituted on carbon by one or more R²²;

R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

or a pharmaceutically acceptable salt, solvate, solvate of such a salt or a prodrug thereof.

Additionally suitable IBAT inhibitor are selected from any one of Example 1-120 of WO 02/50051, or a pharmaceutically acceptable salt, solvate, solvate of such a salt or a prodrug thereof, and the compounds of Examples 1-120 are incorporated herein by reference. Claims 1-14 of WO 02/50051 are also incorporated herein by reference. Particular compounds of formula (AI) are:
1,1-dioxo-3,3-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-sulphoethyl) carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(5-carboxypentyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphorylmethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{2-[(methyl)(ethyl) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N-[(R)-α-(N′-{2-[(methyl)(hydroxy) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[(R)-N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine;
   or a pharmaceutically acceptable salt, solvate, solvate of such a salt or a prodrug thereof.

Additional suitable IBAT inhibitors are those described in WO 03/020710. Further suitable compounds possessing IBAT inhibitory activity have the following structure of formula (BI): 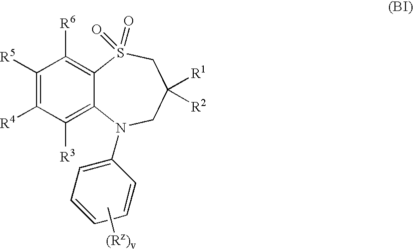
wherein:

One of R¹ and R² are selected from hydrogen or C₁₋₆alkyl and the other is selected from C₁₋₆alkyl;

R^{z} is selected from halo, nitro, cyano, hydroxy, amino, carboxy, carbamoyl, mercapto, sulphamoyl, C₁₋₆alkyl, C₂₋₆alkenyl, C₂ alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁴ and R⁵ is a group of formula (BIA):

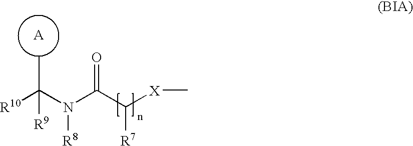

R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl; wherein R³ and R⁶ and the other of R⁴ and R⁵ may be optionally substituted on carbon by one or more R¹⁷;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted on carbon by one or more substituents selected from R¹⁸;

R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;

R⁸ is hydrogen or C₁₋₆alkyl;

R⁶ is hydrogen or C₁₋₆alkyl;

R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹—(C₁₋₁₀alkylene)_{q}- or heterocyclyl-(C₁₋₁₀alkylene)ᵣ-R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (BIB): 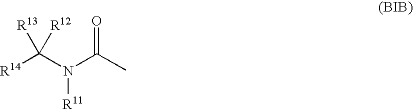
wherein:

R¹¹ is hydrogen or C₁₋₆alkyl;

R¹² and R¹³ are independently selected from hydrogen, halo, nitro, cyano, hydroxy, amino, carbamoyl, mercapto, sulphamoyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁alkyl)amino, N,N-(C₁₋₁₀alkyl)₂amino, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;

R¹⁴ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₄₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (BIC):

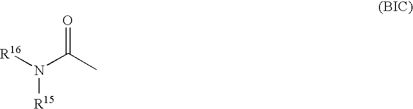

R¹⁵ is hydrogen or C₁₋₆alkyl;

R¹⁶ is hydrogen or C₁₋₆alkyl; wherein R¹⁶ may be optionally substituted on carbon by one or more groups selected from R³¹;

n is 1-3; wherein the values of R⁷ may be the same or different;

R¹⁷, R¹⁸, R¹⁹, R²³, R²⁵, R²⁹ or R³¹ are independently selected from halo, nitro, cyano, hydroxy, amino, carbamoyl, mercapto, sulphamoyl, hydroxyaminocarbonyl, C₁₋₁alkyl, C₂₋₁₀alkenyl, C₂₋₁₀alkyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)₆ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R⁷, R¹⁸, R¹⁹, R²³, R²⁵, R²⁹ or R³¹ may be independently optionally substituted on carbon by one or more R³⁴; and wherein if said heterocyclyl contains an —NH— group, that nitrogen may be optionally substituted by a group selected from R³⁵;

R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;

p, q, r and s are independently selected from 0-2;

R³ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, alkyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;

R²⁰, R²⁴, R²⁶, R³⁰ or R³⁵ are independently selected from C₁₋₁₀alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl; or a pharmaceutically acceptable salt, solvate, solvate of such a salt or a prodrug thereof.

Further suitable IBAT inhibitors are selected from any one of Example 144 of WO 03/020710, or a pharmaceutically acceptable salt, solvate, solvate of such a salt or a prodrug thereof, and the compounds of Examples 1-44 are incorporated herein by reference. Claims 1-10 of WO 03/020710 are also incorporated herein by reference. A particular IBAT inhibitor selected from WO 03/020710 is any one of:
1,1-dioxo-3,3-dibutyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N-((S)-1-carbamoyl-2-hydroxyethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(hydroxycarbamoylmethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N-((R)-α-{N′-[2-(N′-pyrimidin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-[N-((R)-α-{N′-[2-(N-pyridin-2-ylureido)ethyl]carbamoyl}benzyl)carbamoylmethoxy]-2,3,4,5-tetrahydro-1,5-benzothiazepine;
1,1-dioxo-3-butyl-3-ethyl-5-phenyl-7-methylthio-8-(N-{(R)-α-[N′-(1-t-butoxycarbonylpiperidin-4-ylmethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2,3-dihydroxypropyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-[N-((R)-α-{N-[2-(3,4-dihydroxyphenyl)-2-methoxyethyl]carbamoyl}benzyl)carbamoylmethoxy]-2,3,4,5-tetrahydro-1,5-benzothiazepine
1,1-dioxo-3-butyl-3-ethyl-5-phenyl-7-methylthio-8-(N-{(R)-α-[N′-(2-aminoethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(piperidinylmethyl) carbamoyl]benzyl}carbamoylmethoxy)-2,3,4,5-tetrahydro-1,5-benzothiazepine; or
1,1-dioxo-3-butyl-3-ethyl-5-phenyl-7-methylthio-8-(N-{(R)-α-[N′-(2-N,N-dimethylaminosulphamoylethyl)carbamoyl]benzyl}carbamoylmethoxy)-2,3,4,5-tetrahydro-1,5-benzothiazepine;
   or a pharmaceutically acceptable salt, solvate, solvate of such a salt or a prodrug thereof.

Additional suitable IBAT inhibitors are those described in WO 03/022825. Further suitable compounds possessing IBAT inhibitory activity have the following structure of formula (CI): 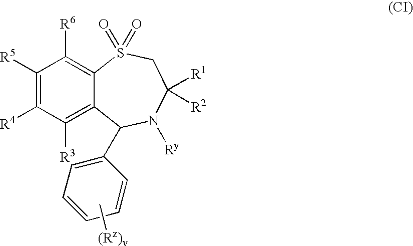
wherein:

One of R¹ and R² are selected from hydrogen or C₁₋₆alkyl and the other is selected from C₁₋₆alkyl;

R^{y} is selected from hydrogen, hydroxy, C₁₋₆alkyl, C₁₋₆alkoxy and C₁₋₆alkanoyloxy;

R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁴ and R⁵ is a group of formula (CIA):

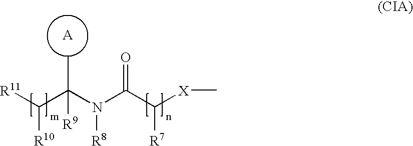

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted by one or more substituents selected from R¹⁷;

R⁷ is hydrogen, C₁₋₄alkyl, carbocyclyl or heterocyclyl; wherein R⁷ is optionally substituted by one or more substituents selected from R¹⁸;

R⁸ is hydrogen or C₁₋₄alkyl;

R⁹ is hydrogen or C₁₋₄alkyl;

R¹⁰ is hydrogen, C₁₋₄alkyl, carbocyclyl or heterocyclyl; wherein R¹⁰ is optionally substituted by one or more substituents selected from R¹⁹;

R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (CIB): 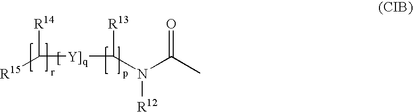
wherein:

Y is —N(R^{x})—, —N(R^{x})C(O)—, —O—, and —S(O)a-; wherein a is 0-2 and R^{x} is hydrogen or C₁₋₄alkyl;

R¹² is hydrogen or C₁₋₄alkyl;

R¹³ and R¹⁴ are independently selected from hydrogen, C₁₋₄alkyl, carbocyclyl or heterocyclyl; wherein R¹³ and R¹⁴ may be independently optionally substituted by one or more substituents selected from R²⁰;

R¹⁵ is carboxy, sulpho, sulphino, phosphono, —P(O)(OR^{e})(OR^{f}), —P(O)(OH)(OR), —P(O)(OH)(R^{e}) or —P(O)(OR^{e})(R^{f}) wherein R^{e} and R^{f} are independently selected from C₁₋₆alkyl;

p is 1-3; wherein the values of R¹³ may be the same or different;

q is 0-1;

r is 0-3; wherein the values of R¹⁴ may be the same or different;

m is 0-2; wherein the values of R¹⁰ may be the same or different;

n is 1-3; wherein the values of R⁷ may be the same or different;

R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;

R¹⁹ and R²⁰ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹ and R²⁰ may be independently optionally substituted on carbon by one or more R²²;

R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

or a pharmaceutically acceptable salt, solvate, solvate of such a salt or a prodrug thereof.

A particular IBAT inhibitor is one selected from Example 1-7 of WO 03/022825, or a pharmaceutically acceptable salt, solvate, solvate of such a salt or a prodrug thereof, and the compounds of Examples 1-7 are incorporated herein by reference. Claims 1-8 of WO 03/022825 are also incorporated herein by reference. A particular IBAT inhibitor selected from WO 03/022825 is any one of:
1,1-dioxo-3(R)-3-butyl-3-ethyl-5-(R)-5-phenyl-8-[N-((R)-α-carboxybenzyl) carbamoylmethoxy]-2,3,4,5-tetrahydro-1,4-benzothiazepine;
1,1-dioxo-3(S)-3-butyl-3-ethyl-5-(S)-5-phenyl-8-[N-((R)-α-carboxybenzyl) carbamoylmethoxy]-2,3,4,5-tetrahydro-1,4-benzothiazepine;
1,1-dioxo-3(R)-3-butyl-3-ethyl-5-(R)-5-phenyl-8-(N-{(R)-α-[N-(carboxymethyl)carbamoyl]benzyl}carbamoylmethoxy)-2,3,4,5-tetrahydro-1,4-benzothiazepine; 1,1-dioxo-3(S)-3-butyl-3-ethyl-5-(S)-5-phenyl-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-bromo-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-(S)-3-ethyl-3-butyl-4-hydroxy-5-(S)-5-phenyl-7-bromo-8-(N-{(R)-α-[N-(carboxymethyl)carbamoyl]benzyl}carbamoylmethoxy)-2,3,4,5-tetrahydro-1,4-benzothiazepine
3,5-trans-1,1-dioxo-3-(R)-3-ethyl-3-butyl-4-hydroxy-5-(R)-5-phenyl-7-bromo-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N-{(R)-α-[N-(carboxymethyl)carbamoyl]benzyl}carbamoylmethoxy)-2,3,4,5-tetrahydro-1,4-benzothiazepine;
3,5-trans-1,1-dioxo-3-ethyl-3-butyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,4-benzothiazepine ammonia salt;
1,1-dioxo-3-(S)-3-ethyl-3-butyl-5-(S)-5-phenyl-7-methylthio-8-(N-{(R)-α-[N-(carboxymethyl)carbamoyl]benzyl}carbamoylmethoxy)-2,3,4,5-tetrahydro-1,4-benzothiazepine diethylamine salt; and
1,1-dioxo-3-(R)-3-ethyl-3-butyl-5-(R)-5-phenyl-7-methylthio-8-(N-{(R)-α-[N-(carboxymethyl)carbamoyl]benzyl}carbamoylmethoxy)-2,3,4,5-tetrahydro-1,4-benzothiazepine diethylamine salt;
   or a pharmaceutically acceptable salt, solvate, solvate of such a salt or a prodrug thereof.

Additional IBAT inhibitors are those described in WO 03/022830. Further suitable compounds possessing IBAT inhibitory activity have the following structure of formula (DI): 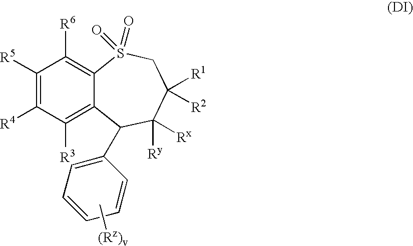
wherein:

One of R¹ and R² are selected from hydrogen or C₁₋₆alkyl and the other is selected from C₁₋₆alkyl;

R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;

R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁴ and R⁵ is a group of formula (DIA):

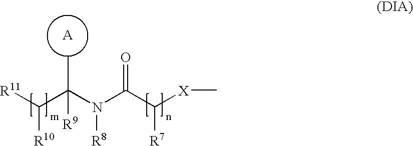

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted by one or more substituents selected from R¹⁷;

R⁷ is hydrogen, C₁₋₄alkyl, carbocyclyl or heterocyclyl; wherein R⁷ is optionally substituted by one or more substituents selected from R¹⁸;

R⁸ is hydrogen or C₁₋₄alkyl;

R⁹ is hydrogen or C₁₋₄alkyl;

R¹⁰ is hydrogen, C₁₋₄alkyl, carbocyclyl or heterocyclyl; wherein R¹⁰ is optionally substituted by one or more substituents selected from R¹⁹;

R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (DIB): 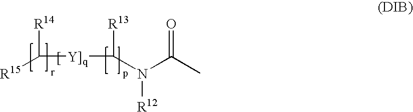
wherein:

Y is —N(Rⁿ)—, —N(Rⁿ)C(O)—, —O—, and —S(O)a-; wherein a is 0-2 and Rⁿ is hydrogen or C₁₋₄alkyl;

R¹² is hydrogen or C₁₋₄alkyl;

R¹³ and R¹⁴ are independently selected from hydrogen, C₁₋₄alkyl, carbocyclyl or heterocyclyl; wherein R¹³ and R¹⁴ may be independently optionally substituted by one or more substituents selected from R²⁰;

R¹⁵ is carboxy, sulpho, sulphino, phosphono, —P(O)(OR^{e})(OR^{f}), —P(O)(OH)(OR^{e}), P(O)(OH)(R^{e}) or —P(O)(OR^{e})(R^{f}) wherein R^{e} and R^{f} are independently selected from C₁₋₆alkyl;

p is 1-3; wherein the values of R¹³ may be the same or different;

q is 0-1;

r is 0-3; wherein the values of R¹⁴ may be the same or different;

m is 0-2; wherein the values of R¹⁰ may be the same or different;

n is 1-3; wherein the values of R⁷ may be the same or different;

R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁸ may be independently optionally substituted on carbon by one or more R²¹;

R¹⁹ and R²⁰ are independently selected from halo, nitro, cyano, hydroxy, amino, carboxy, carbamoyl, mercapto, sulphamoyl, C₁₋₄alkyl, C₂₋₄alkenyl, C₂-alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹ and R²⁰ may be independently optionally substituted on carbon by one or more R²²;

R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

or a pharmaceutically acceptable salt, solvate, solvate of such a salt or a prodrug thereof.

A particular IBAT inhibitor is selected from any one of Example 14 of WO 03/022830, or a pharmaceutically acceptable salt, solvate, solvate of such a salt or a prodrug thereof, and the compounds of Examples 14 are incorporated herein by reference. Claims 1-8 of WO 03/022830 are also incorporated herein by reference. A IBAT inhibitor selected from WO 03/022830 is any one of:
1,1-dioxo-3-butyl-3-ethyl-4-hydroxy-5-phenyl-7-(N-{(R)-α-[N-(carboxymethyl)carbamoyl]benzyl}carbamoylmethylthio)-2,3,4,5-tetrahydrobenzothiepine
1,1-dioxo-3-butyl-3-ethyl-4-hydroxy-5-phenyl-7-(N-{(R)-α-[N-(2-sulphoethyl)carbamoyl]-4-hydroxybenzyl}carbamoylmethylthio)-2,3,4,5-tetrahydrobenzothiepine ammonia salt
1,1-dioxo-3-butyl-3-ethyl-4-hydroxy-5-phenyl-7-{N-[α-(carboxy)-2-fluorobenzyl]carbamoylmethylthio}-2,3,4,5-tetrahydrobenzothiepine; and
1,1-dioxo-3-butyl-3-ethyl-4-hydroxy-5-phenyl-7-{N-[1-(carboxy)-1-(thien-2-yl)methyl]carbamoylmethylthio}-2,3,4,5-tetrahydrobenzothiepine
   or a pharmaceutically acceptable salt, solvate, solvate of such a salt or a prodrug thereof.

Additional suitable IBAT inhibitors are those described in WO 03/022286. Further suitable compounds possessing IBAT inhibitory activity have the following structure of formula (EI): 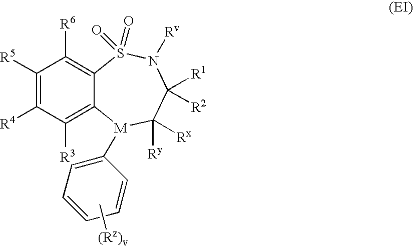
wherein:

R^{v} is selected from hydrogen or C₁₋₆alkyl;

One of R¹ and R² are selected from hydrogen or C₁₋₆alkyl and the other is selected from C₁₋₆alkyl;

R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;

M is selected from —N— or —CH—;

R^{z} is selected from halo, nitro, cyano, hydroxy, amino, carboxy, carbamoyl, mercapto, sulphamoyl, C₁₋₆alkyl, C₁₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁴ and R⁵ is a group of formula (EIA):

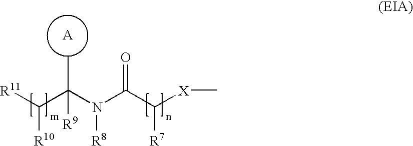

R³ and R⁶ and the other of R⁴ and R⁵ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₄₋alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R³ and R⁶ and the other of R⁴ and R⁵ may be optionally substituted on carbon by one or more R¹⁶;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted by one or more substituents selected from R¹⁷;

R⁷ is hydrogen, C₁₋₄alkyl, carbocyclyl or heterocyclyl; wherein R⁷ is optionally substituted by one or more substituents selected from R¹⁸;

R⁸ is hydrogen or C₁₋₄alkyl;

R⁹ is hydrogen or C₁₋₄alkyl;

R¹⁰ is hydrogen, C₁₋₄alkyl, carbocyclyl or heterocyclyl; wherein R¹⁰ is optionally substituted by one or more substituents selected from R¹⁹;

R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆alkyl; or R¹¹ is a group of formula (EIB) or (EIC): 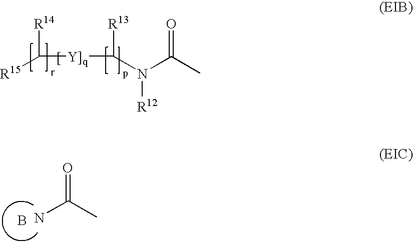
wherein:

Y is —N(Rⁿ)—, —N(Rⁿ)C(O)—, —N(Rⁿ)C(O)(CR^{s}R^{t})ᵥN(Rⁿ)C(O)—, —O—, and —S(O)a-; wherein a is 0-2, v is 1-2, R^{s} and R^{t} are independently selected from hydrogen or C₁₋₄alkyl optionally substituted by R²⁶ and Rⁿ is hydrogen or C₁₋₄alkyl;

R¹² is hydrogen or C₁₋₄alkyl;

R¹³ and R¹⁴ are independently selected from hydrogen, C₁₋₄alkyl, carbocyclyl or heterocyclyl; and when q is 0, R¹⁴ may additionally be selected from hydroxy; wherein R¹³ and R¹⁴ may be independently optionally substituted by one or more substituents selected from R²⁰;

R¹⁵ is carboxy, sulpho, sulphino, phosphono, —P(O)(OR^{e})(OR^{f}), —P(O)(OH)(OR^{e}), —P(O)(OH)(R^{e}) or —P(O)(OR(R^{f}) wherein R^{e} and R^{f} are independently selected from C₁₋₆alkyl;

p is 1-3; wherein the values of R¹³ may be the same or different;

q is 0-1;

r is 0-3; wherein the values of R¹⁴ may be the same or different;

m is 0-2; wherein the values of R¹⁰ may be the same or different;

n is 1-3; wherein the values of R⁷ may be the same or different;

Ring B is a nitrogen linked heterocyclyl substituted on carbon by one group selected from R²³, and optionally additionally substituted on carbon by one or more R²⁴; and wherein if said nitrogen linked heterocyclyl contains an —NH— moiety, that nitrogen may be optionally substituted by a group selected from R²⁵;

R¹⁶, R¹⁷ and R¹⁸ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R¹⁶, R¹⁷ and R¹⁵ may be independently optionally substituted on carbon by one or more R²¹;

R¹⁹, R²⁰, R²⁴ and R²⁶ are independently selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N-(C₁₋₄alkyl)₂sulphamoyl, carbocyclyl, heterocyclyl, benzyloxycarbonylamino, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆alkyl; wherein R¹⁹, R²⁰, R²⁴ and R²⁶ may be independently optionally substituted on carbon by one or more R²²;

R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl;

R²³ is carboxy, sulpho, sulphino, phosphono, —P(O)(OR^{g})(OR^{h}), —P(O)(OH)(OR^{g}), —P(O)(OH)(R^{g}) or —P(O)(OR^{g})(R^{h}) wherein R^{g} and R^{h} are independently selected from C₁₋₆alkyl;

R²⁵ is selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;

or a pharmaceutically acceptable salt, solvate, solvate of such a salt or a prodrug thereof.

A particular IBAT inhibitor is selected from any one of Example 1-39 of WO 03/022286, or a pharmaceutically acceptable salt, solvate, solvate of such a salt or a prodrug thereof, and the compounds of Examples 1-39 are incorporated herein by reference. Claims 1-10 of WO 03/022286 are also incorporated herein by reference. A IBAT inhibitor selected from WO 03/022286 is any one of:
1,1-dioxo-3,3-dibutyl-5-phenyl-7-methylthio-8-(N-{(R)-α-[N-((R)-1-carboxy-2-methylthio-ethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxybutyl) carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl)carbamoyl]hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((R)-1-carboxy-2-methylthioethyl)carbamoyl)benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-{(S)-1-[N-((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-[N-((R)-α-carboxy-4-hydroxybenzyl)carbamoylmethoxy]-2,3,4,5-tetrahydro-1,2,5-benzothiadiazepine;
   or a pharmaceutically acceptable salt, solvate, solvate of such a salt or a prodrug thereof.

Further suitable compounds possessing IBAT inhibitory activity have the following structure of formula (FFI): 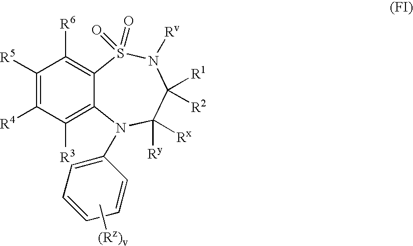
wherein:

R^{v} is selected from hydrogen or C₁₋₆alkyl;

One of R¹ and R² are selected from hydrogen or C₁₋₆alkyl and the other is selected from C₁₋₆alkyl;

R^{x} and R^{y} are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;

R^{z} is selected from halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁴ and R⁵ is a group of formula (FIA):

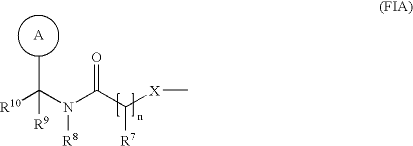

R³ and R⁶ and the other of R⁴ and R⁵ are independently selected from hydrogen, halo, nitro, cyano, hydroxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl; wherein R³ and R⁶ and the other of R⁴ and R⁵ may be optionally substituted on carbon by one or more R¹⁷;

X is —O—, —N(R^{a})—, —S(O)_{b}— or —CH(R^{a})—; wherein R^{a} is hydrogen or C₁₋₆alkyl and b is 0-2;

Ring A is aryl or heteroaryl; wherein Ring A is optionally substituted on carbon by one or more substituents selected from R¹⁸;

R⁷ is hydrogen, C₁₋₆alkyl, carbocyclyl or heterocyclyl; wherein R⁷ is optionally substituted on carbon by one or more substituents selected from R¹⁹; and wherein if said heterocyclyl contains an —NH— group, that nitrogen may be optionally substituted by a group selected from R²⁰;

R⁸ is hydrogen or C₁₋₆alkyl;

R⁹ is hydrogen or C₁₋₆alkyl;

R¹⁰ is hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²¹—(C₁₋₁₀alkylene)_{q}- or heterocyclyl-(C₁₋₁₀alkylene)ᵣ-R²²—(C₁₋₁₀alkylene)ₛ-; wherein R¹⁰ is optionally substituted on carbon by one or more substituents selected from R²³; and wherein if said heterocyclyl contains an —NH— group, that nitrogen may be optionally substituted by a group selected from R²⁴; or R¹⁰ is a group of formula (FIB): 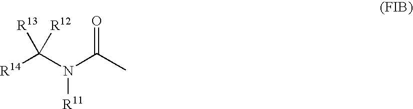
wherein:

R¹¹ is hydrogen or C₁₋₆alkyl;

R¹² and R¹³ are independently selected from hydrogen, halo, carbamoyl, sulphamoyl, C₁₋₁₀alkyl, C₂₋₁₀alkenyl, C₂₋₁₀alkynyl, C₁₋₁₀alkanoyl, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl or heterocyclyl; wherein R¹² and R¹³ may be independently optionally substituted on carbon by one or more substituents selected from R²⁵; and wherein if said heterocyclyl contains an —NH— group, that nitrogen may be optionally substituted by a group selected from R²⁶;

R¹⁴ is selected from hydrogen, halo, carbamoyl, sulphamoyl, hydroxyaminocarbonyl, C₁₋₁₀alkyl, C₂₋₁₀alkenyl, C₂₋₁₀alkynyl, C₁₋₁₀alkanoyl, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclyl, heterocyclylC₁₋₁₀alkyl, carbocyclyl-(C₁₋₁₀alkylene)ₚ-R²⁷—(C₁₋₁₀alkylene)_{q}- or heterocyclyl-(C₁₋₁₀alkylene)ᵣ-R²⁸—(C₁₋₁₀alkylene)ₛ-; wherein R¹⁴ may be optionally substituted on carbon by one or more substituents selected from R²⁹; and wherein if said heterocyclyl contains an —NH— group, that nitrogen may be optionally substituted by a group selected from R³⁰; or R¹⁴ is a group of formula (FIC):

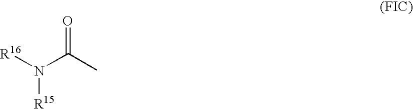

R¹⁵ is hydrogen or C₁₋₆alkyl;

R¹⁶ is hydrogen or C₁₋₆alkyl; wherein R¹⁶ may be optionally substituted on carbon by one or more groups selected from R³¹;

n is 1-3; wherein the values of R⁷ may be the same or different;

R¹⁷, R¹⁸, R¹⁹, R²⁰, R²⁵, R²⁹ or R³¹ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclyl, heterocyclylC₁₋₁₀alkyl, carbocyclyl-(C₁₋₁₀alkylene)ₚ-R³²—(C₁₋₁₀alkylene)_{q}- or heterocyclyl-(C₁₋₁₀alkylene)ᵣ-R³³—(C₁₋₁₀alkylene)ₛ-; wherein R⁷, R¹⁵, R¹⁹, R²³, R², R²⁹ or R³¹ may be independently optionally substituted on carbon by one or more R³⁴; and wherein if said heterocyclyl contains an —NH— group, that nitrogen may be optionally substituted by a group selected from R³⁵;

R²¹, R²², R²⁷, R²⁸, R³² or R³³ are independently selected from —O—, —NR³⁶—, —S(O)ₓ—, —NR³⁶C(O)NR³⁶—, —NR³⁶C(S)NR³⁶—, —OC(O)N═C—, —NR³⁶C(O)— or —C(O)NR³⁶—; wherein R³⁶ is selected from hydrogen or C₁₋₆alkyl, and x is 0-2;

p, q, r and s are independently selected from 0-2;

R³⁴ is selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, alkyl, ethynyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl, N,N-dimethylsulphamoyl, N-methylsulphamoylamino and N,N-dimethylsulphamoylamino;

R²⁰, R²⁴, R²⁶, R³⁰ or R³⁵ are independently selected from C₁₋₆alkyl, C₁₋₆alkanoyl, C₁₋₆alkylsulphonyl, C₁₋₆alkoxycarbonyl, carbamoyl, N-(C₁₋₆alkyl)carbamoyl, N,N-(C₁₋₆alkyl)carbamoyl, benzyl, benzyloxycarbonyl, benzoyl and phenylsulphonyl;

or a pharmaceutically acceptable salt, solvate, solvate of such a salt or a prodrug thereof.

Suitable IBAT inhibitors having the above structure are selected from any one of:
1,1-Dioxo-3,3-dibutyl-5-phenyl-7-methylthio-8-(N-{(R)-α-[N-(2-(S)-3-(R)-4-(R)-5-(R)-2,3,4,5,6-pentahydroxyhexyl)carbamoyl]benzyl}carbamoylmethoxy)-2,3,4,5-tetrahydro-1,2,5-benzothiadiazepine;
1,1-Dioxo-3,3-dibutyl-5-phenyl-7 methylthio-8-(N-{(R)-α-[N-(2-(S)-3-(R)-4-(R)-5-(R)-2,3,4,5,6-pentahydroxyhexyl)carbamoyl]-4-hydroxybenzyl}carbamoylmethoxy)-2,3,4,5-tetrahydro-1,2,5-benzothiadiazepine;
1,1-Dioxo-3,3-dibutyl-5-phenyl-7-methylthio-8-[N-((R/S)-α-{N-[1-(R)-2-(S)-1-hydroxy-1-(3,4-dihydroxyphenyl)prop-2-yl]carbamoyl}benzyl)carbamoylmethoxy]-2,3,4,5-tetrahydro-1,2,5-benzothiadiazepine (both enantiomers);
1,1-Dioxo-3,3-dibutyl-5-phenyl-7-methylthio-8-{N-[(R)-α-(N-{2-(S)-[N-(carbamoylmethyl) carbamoyl]pyrrolidin-1-ylcarbonylmethyl}carbamoyl)benzyl]carbamoylmethoxy}-2,3,4,5-tetrahydro-1,2,5-benzothiadiazepine;
1,1-Dioxo-3,3-dibutyl-5-phenyl-7-methylthio-8-[N-((R)-α-{N-[2-(3,4,5-trihydroxyphenyl)ethyl]carbamoyl}benzyl)carbamoylmethoxy]-2,3,4,5-tetrahydro-1,2,5-benzothiadiazepine; or
1,1-Dioxo-3,3-dibutyl-5-phenyl-7-methylthio-8-(N-{(R)-α-[N-(2-(R)-3-(S)-4-(S)-5-(R)-3,4,5,6-tetrahydroxytetrahydropyran-2-ylmethyl)carbamoyl]benzyl}carbamoylmethoxy)-2,3,4,5-tetrahydro-1,2,5-benzothiadiazepine;
   or a pharmaceutically acceptable salt, solvate, solvate of such a salt or a prodrug thereof.

Further suitable IBAT inhibitors include a compound of formula (GI): 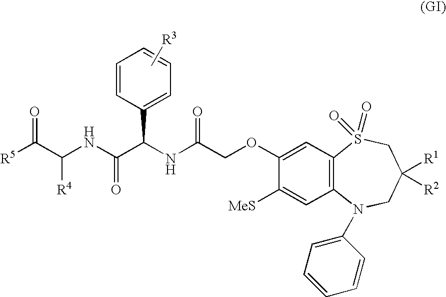
wherein:

R¹ and R² are independently selected from C₁₋₄alkyl;

R³ is hydrogen, hydroxy or halo;

R⁴ is C₁₋₄alkyl optionally substituted by hydroxy, methoxy and methylS(O)ₐ wherein a is 0-2

R⁵ is hydroxy or HOC(O)CH(R⁶)NH—;

R⁶ is selected from hydrogen and C₁₋₃alkyl optionally substituted by hydroxy, methoxy and methylS(O)ₐ wherein a is 0-2;

or a pharmaceutically acceptable salt, solvate, solvate of such a salt or a prodrug thereof; with the proviso that when R¹ and R² are both butyl, R⁵ is hydroxy and R⁴ is methylthiomethyl, methylsulphinylmethyl, 2-methylthioethyl, hydroxymethyl, methoxymethyl; R³ is not hydrogen; and with the proviso that when R¹ and R² are both butyl, R⁵ is HOC(O)CH(R⁶)NH—, R⁶ is hydroxymethyl and R⁴ is hydroxymethyl; R³ is not hydrogen.

Suitable IBAT inhibitors having the above structure are selected from any one of:
1,1-dioxo-3,3-dibutyl-5-phenyl-7-methylthio-8-(N-{(R)-α-[N′-((S)-1-carboxyethyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxypropyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxybutyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylbutyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-hydroxypropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-mesylethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylsulphonylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-mesylpropyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxyethyl) carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxypropyl) carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxybutyl) carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylpropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylbutyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylbutyl)carbamoyl]hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-hydroxyethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-hydroxypropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylthioethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methylsulphinylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-mesylethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-2-methoxyethyl)carbamoyl]hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-(N′-((S)-1-carboxy-3-methylthiopropyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-((S)-1-carboxy-3-methylsulphonylpropyl)carbamoyl]-4-hydroxybenzyl}carbamoylmethoxy)-2,3,4,5-tetrahydro-1,5-benzothiazepine; or
1,1-dioxo-3,3-dibutyl-5-phenyl-7-methylthio-8-(N-{(R)-α-[N′-((S)-1-carboxy-3-mesylpropyl)carbamoyl]-4-hydroxybenzyl}carbamoylmethoxy)-2,3,4,5-tetrahydro-1,5-benzothiazepine;
   or a pharmaceutically acceptable salt, solvate, solvate of such a salt or a prodrug thereof.

Additional suitable IBAT inhibitors having the above structure are selected from:
1,1-dioxo-3,3-dibutyl-5-phenyl-7-methylthio-8-(N-{(R)-α-[N′-((S)-1-carboxypropyl)carbamoyl]-4-hydroxybenzyl}carbamoylmethoxy)-2,3,4,5-tetrahydro-1,5-benzothiazepine; or
1,1-dioxo-3,3-dibutyl-5-phenyl-7-methylthio-8-(N-{(R)-α-[N′-((S)-1-carboxyethyl) carbamoyl]benzyl}carbamoylmethoxy)-2,3,4,5-tetrahydro-1,5-benzothiazepine.

Further suitable IBAT inhibitors are those having the structure (HI): 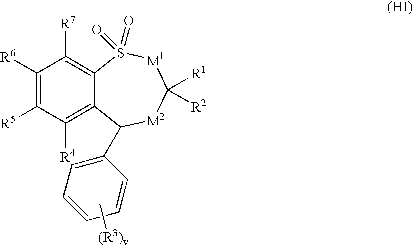
wherein

M¹ is —CH₂— or —NR²¹—;

M² is —CR²²R²³— or —NR²⁴—; provided that if M¹ is —NR²¹—, M² is —CR²²R²³—;

One of R¹ and R² are selected from hydrogen, C₁₋₆alkyl or C₂₋₆alkenyl and the other is selected from C₁₋₆alkyl or C₂₋₆alkenyl;

R³ is selected from halo, nitro, cyano, hydroxy, amino, carboxy, carbamoyl, mercapto, sulphamoyl, C₁₋₆alkyl, C₂₋₆alkenyl, C₂ alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl and N,N-(C₁₋₆alkyl)₂sulphamoyl;

v is 0-5;

one of R⁵ and R⁶ is a group of formula (MA):

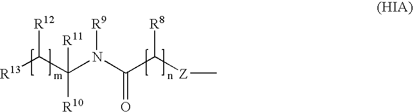

R⁴ and R⁷ and the other of R⁵ and R⁶ are independently selected from hydrogen, halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R⁴ and R⁷ and the other of R⁵ and R⁶ may be optionally substituted on carbon by one or more R²⁵;

Z is —O—, —N(R^{a})—, —S(O)_{b}— or —CH(R^{a})—; wherein R^{a} is hydrogen or C₁₋₆alkyl and b is 0-2;

R⁸ is hydrogen, C₁₋₄ alkyl, carbocyclyl or heterocyclyl; wherein R⁸ may be optionally substituted on carbon by one or more substituents selected from R²⁶; and wherein if said heterocyclyl contains an —NH— group, that nitrogen may be optionally substituted by a group selected from R²⁷;

R⁹ is hydrogen or C₁₋₄alkyl;

R¹⁰ and R¹¹ are independently selected from hydrogen, C₁₋₄alkyl, carbocyclyl or heterocyclyl; or R¹⁰ and R¹¹ together form C₂₋₆alkylene; wherein R¹⁰ and R¹¹ or R¹⁰ and R¹¹ together may be independently optionally substituted on carbon by one or more substituents selected from R²⁸; and wherein if said heterocyclyl contains an —NH— moiety, that nitrogen may be optionally substituted by one or more R²⁹;

R¹² is hydrogen, C₁₋₄alkyl, carbocyclyl or heterocyclyl; wherein R¹² may be optionally substituted on carbon by one or more substituents selected from R³⁰; and wherein if said heterocyclyl contains an —NH— moiety, that nitrogen may be optionally substituted by one or more R³¹;

R¹³ is hydrogen, halo, nitro, cyano, hydroxy, amino, carbamoyl, mercapto, sulphamoyl, hydroxyaminocarbonyl, C₁₋₁₀ alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclic group, heterocyclylC₁₋₀alkyl, carbocyclyl-(C₁₋₀alkylene)ₑ-R³²—(C₁₋₁₀alkylene)_{f}- or heterocyclyl-(C₁₋₁₀alkylene)_{g}-R³³—(C₁₋₁₀alkylene)ₕ-; wherein R¹³ may be optionally substituted on carbon by one or more substituents selected from R³⁶; and wherein if said heterocyclyl contains an —NH— group, that nitrogen may be optionally substituted by a group selected from R³⁷; or R¹³ is a group of formula (HIB): 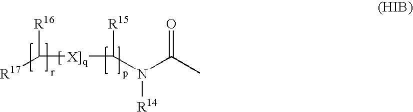
wherein:

X is —N(R³⁸)—, —N(R³⁸)C(O)—, —O—, and —S(O)ₐ—; wherein a is 0-2 and R³⁸ is hydrogen or C₁₋₄alkyl;

R¹⁴ is hydrogen or C₁₋₄alkyl;

R¹⁵ and R¹⁶ are independently selected from hydrogen, halo, nitro, cyano, hydroxy, amino, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl or heterocyclic group; wherein R¹⁵ and R¹⁶ may be independently optionally substituted on carbon by one or more substituents selected from R⁴¹; and wherein if said heterocyclyl contains an —NH— group, that nitrogen may be optionally substituted by a group selected from R⁴²;

R¹⁷ is selected from hydrogen, halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, N-(C₁₋₁₀alkyl)amino, N,N-(C₁₋₁₀alkyl)₂amino, C₁₋₁₀alkanoylamino, N-(C₁₋₁₀alkyl)carbamoyl, C₁₋₁₀alkoxycarbonyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, carbocyclyl, carbocyclylC₁₋₁₀alkyl, heterocyclic group, heterocyclylC₁₋₁₀alkyl, carbocyclyl-(C₁₋₁₀alkylene)ₑ-R⁴³—(C₁₋₁₀alkylene)_{f}- or heterocyclyl-(C₁₋₁₀alkene)_{g}-R⁴⁴—(C₁₋₁₀alkylene)ₕ-; wherein R¹⁷ may be optionally substituted on carbon by one or more substituents selected from R⁴⁷; and wherein if said heterocyclyl contains an —NH— group, that nitrogen may be optionally substituted by a group selected from R⁴⁸; or R¹⁷ is a group of formula (HIC): 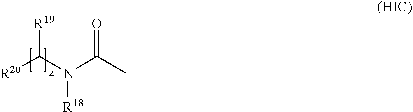
wherein:

R¹⁸ is selected from hydrogen or C₁₋₄alkyl;

R¹⁹ is selected from hydrogen, halo, nitro, cyano, hydroxy, amino, carbamoyl, mercapto, sulphamoyl, C₁₋₆alkyl, C₂₋₆alkenyl, C₂₋₆alkyl, C₁₋₆alkoxy, C₁₋₆alkanoyl, C₁₋₆alkanoyloxy, N-(C₁₋₆alkyl)amino, N,N-(C₁₋₆alkyl)₂amino, C₁₋₆alkanoylamino, N-(C₁₋₆alkyl)carbamoyl, N,N-(C₁₋₆alkyl)₂carbamoyl, C₁₋₆alkylS(O)ₐ wherein a is 0 to 2, C₁₋₆alkoxycarbonyl, N-(C₁₋₆alkyl)sulphamoyl, N,N-(C₁₋₆alkyl)₂sulphamoyl, carbocyclyl or heterocyclic group; where R¹⁹ may be independently optionally substituted on carbon by one or more substituents selected from R⁵¹; and wherein if said heterocyclyl contains an —NH— group, that nitrogen may be optionally substituted by a group selected from R⁵²;

R²⁰ is selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkoxycarbonyl, C₁₋₁₀alkanoyl, C₁₋₁₀alkanoyloxy, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclic group, heterocyclylC₁₋₁₀alkyl, carbocyclyl-(C₁₋₁₀alkylene)ₑ-R⁵³—(C₁₋₁₀alkylene)_{f}- or heterocyclyl-(C₁₋₁₀alkylene)_{g}-R⁵⁴—(C₁₋₁₀alkylene)ₕ-; wherein R²⁰ may be independently optionally substituted on carbon by one or more R⁵⁷; and wherein if said heterocyclyl contains an —NH— group, that nitrogen may be optionally substituted by a group selected from R⁵⁸;

p is 1-3; wherein the values of R¹⁵ may be the same or different;

q is 0-1;

r is 0-3; wherein the values of R¹⁶ may be the same or different;

m is 0-2; wherein the values of R¹² may be the same or different;

n is 1-2; wherein the values of R⁸ may be the same or different;

z is 0-3; wherein the values of R¹⁹ may be the same or different;

R²¹ is selected from hydrogen or C₁₋₆alkyl;

R²² and R²³ are independently selected from hydrogen, hydroxy, amino, mercapto, C₁₋₆alkyl, C₁₋₆alkoxy, N-(C₁₋₆alkyl)amino, N,N-(C₁₋₆alkyl)₂amino, C₁₋₆alkylS(O)ₐ wherein a is 0 to 2;

R²⁴ is selected from hydrogen, hydroxy, C₁₋₆alkyl, C₁₋₄alkoxy and C₁₋₆alkanoyloxy;

R²⁵ is selected from halo, nitro, cyano, hydroxy, amino, carboxy, carbamoyl, mercapto, sulphamoyl, C₁₋₄alkyl, C₂₋₄alkenyl, C₂₋₄alkyl, C₁₋₄alkoxy, C₁₋₄alkanoyl, C₁₋₄alkanoyloxy, N-(C₁₋₄alkyl)amino, N,N-(C₁₋₄alkyl)₂amino, C₁₋₄alkanoylamino, N-(C₁₋₄alkyl)carbamoyl, N,N-(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl and N,N-(C₁₋₄alkyl)₂sulphamoyl; wherein R²⁵, may be independently optionally substituted on carbon by one or more R⁶⁷;

R²⁶, R²⁷, R³⁰, R³⁶, R⁴¹, R⁴⁷, R⁵¹ and R⁵⁷ are independently selected from halo, nitro, cyano, hydroxy, amino, carbamoyl, mercapto, sulphamoyl, hydroxyaminocarbonyl, C₁₋₁₀alkyl, C₂₋₁₀alkenyl, C₂₋₁₀alkynyl, C₁₋₁₀alkoxy, C₁₋₁₀alkanoyl, C₁₋₁₀alkanoyloxy, C₁₋₁₀alkoxycarbonyl, N-(C₁₋₁₀alkyl)amino, N,N-(C₁₋₁₀alkyl)₂amino, N,N,N-(C₁₋₁₀alkyl)₃ammonio, C₁₋₁₀alkanoylamino, N-(C₁₋₁₀alkyl)carbamoyl, N,N-(C₁₋₁₀alkyl)₂carbamoyl, C₁₋₁₀alkylS(O)ₐ wherein a is 0 to 2, N-(C₁₋₁₀alkyl)sulphamoyl, N,N-(C₁₋₁₀alkyl)₂sulphamoyl, N-(C₁₋₁₀alkyl)sulphamoylamino, N,N-(C₁₋₁₀alkyl)₂sulphamoylamino, C₁₋₁₀alkoxycarbonylamino, carbocyclyl, carbocyclylC₁₋₁₀alkyl, heterocyclic group, heterocyclylC₁₋₁₀alkyl, carbocyclyl-(C₁₋₁₀alkylene)ₑ-R⁵⁹—(C₁₋₁₀alkylene)_{f}- or heterocyclyl-(C₁₋₁₀alkylene)_{g}-R⁶⁰—(C₁₋₁₀alkylene)ₕ-; wherein R²⁶, R²⁸, R³⁰, R³⁶, R⁴¹, R⁴⁷, R⁵¹ and R⁵⁷ may be independently optionally substituted on carbon by one or more R⁶³; and wherein if said heterocyclyl contains an —NH— group, that nitrogen may be optionally substituted by a group selected from R⁶⁴;

R²⁷, R²⁹, R³¹, R³⁷, R⁴², R⁴⁸, R⁵², R⁵⁸ and R⁶⁴ are independently selected from C₁₋₆alkyl, C₁₋₆alkanoyl, C₁₋₆alkylsulphonyl, sulphamoyl, N-(C₁₋₆alkyl)sulphamoyl, N,N-(C₁₋₆alkyl)₂sulphamoyl, C₁₋₆alkoxycarbonyl, carbamoyl, N-(C₁₋₆alkyl)carbamoyl, N,N-(C₁₋₆alkyl)₂carbamoyl, benzyl, phenethyl, benzoyl, phenylsulphonyl and phenyl;

R³², R³³, R⁴³, R⁴⁴, R⁵³, R⁵⁴, R⁵⁹ and R⁶⁰ are independently selected from —O—, —NR⁶⁵—, —S(O)ₓ, —NR⁶⁵C(O)NR⁶⁶—, —NR⁶⁵C(S)NR⁶⁶—, —OC(O)N═C, —NR⁶⁵C(O)— or —C(O)NR⁶⁵—; wherein R⁶⁵ and R⁶⁶ are independently selected from hydrogen or C₁₋₆alkyl, and x is 0-2;

R⁶³ and R⁶⁷ are independently selected from halo, hydroxy, cyano, carbamoyl, ureido, amino, nitro, carbamoyl, mercapto, sulphamoyl, trifluoromethyl, trifluoromethoxy, methyl, ethyl, methoxy, ethoxy, vinyl, alkyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl; and

e, f, g and h are independently selected from 0-2;

or a pharmaceutically acceptable salt, solvate, solvate of such a salt or a prodrug thereof.

Additional suitable IBAT inhibitors having the above structure are selected from any one of:
(+/−)-trans-1,1-dioxo-3-ethyl-3-butyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,4-benzothiazepine;
(+/−)-trans-1,1-dioxo-3-ethyl-3-butyl-5-phenyl-7-methylthio-8-(N-{(R)-α-[N′-(2-(S)-3-(R)-4-(R)-5-(R)-2,3,4,5,6-pentahydroxyhexyl)carbamoyl]benzyl}carbamoylmethoxy)-2,3,4,5-tetrahydro-1,4-benzothiazepine;
1,1-dioxo-3-ethyl-3-butyl-4-hydroxy-5-phenyl-7-(N-{α-[N′-(2-(S)-3-(R)-4-(R)-5-R)-2,3,4,5,6-pentahydroxyhexyl)carbamoyl]-2-fluorobenzyl}carbamoylmethylthio)-2,3,4,5-tetrahydrobenzothiapine; or
1,1-dioxo-3-butyl-3-ethyl-4-hydroxy-5-phenyl-7-(N-1{-[N′-(2-(S)-3-(R)-4-(R)-5-(R)-2,3,4,5,6-pentahydroxyhexyl)carbamoyl]-1-(cyclohexyl)methyl}carbamoylmethylthio)-2,3,4,5-tetrahydrobenzothiepine.

Compounds of formula (AI), (BI), (CI), (DI), (EI), (FI), (GI) and (HI) or a pharmaceutically acceptable salt, solvate, solvate of such a salt or a prodrug thereof may be prepared by processes known in the art.

In a particular aspect of the invention an IBAT inhibitor or a pharmaceutically acceptable salt, solvate, solvate of such a salt or a prodrug thereof is an IBAT inhibitor or a pharmaceutically acceptable salt thereof.

Suitable pharmaceutically acceptable salts of the above compounds, or other compounds disclosed herein, are, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric, acetate or maleic acid. In addition a suitable pharmaceutically acceptable salt of a compound which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The IBAT inhibitor compounds disclosed herein may be administered in the form of a pro-drug which is broken down in the human or animal body to give the parent compound. Examples of pro-drugs include in vivo hydrolysable esters and in vivo hydrolysable amides.

An in vivo hydrolysable ester of a compound containing carboxy or hydroxy group is, for example, a pharmaceutically acceptable ester which is hydrolysed in the human or animal body to produce the parent acid or alcohol. Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds.

An in vivo hydrolysable ester of a compound containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

A suitable value for an in vivo hydrolysable amide of a compound containing a carboxy group is, for example, a N-C₁₋₆alkyl or N,N-di-C₁₋₆alkyl amide such as N-methyl, N-ethyl, N-propyl, N,N-dimethyl, N-ethyl-N-methyl or N,N-diethyl amide.

### EXPERIMENTAL

The following four in vitro examples (Examples A-D) illustrate how calcium salts may be used for lowering the bile salt concentrations in aqueous solutions. These experiments illustrate the underlying mechanism for bile acid sequestering in vivo.

### Example A

### Reduction of the Concentration of Taurocholic Acid in Simulated Intestinal Fluid Caused by Addition of Calcium Chloride

A solution simulating the human intestinal fluid in the fasted state, FaSSIF, was prepared by dissolving the following components in deionised water.

| | |
|---|---|
| Sodium taurocholate | 3.1 mM |
| E-phosphatidylcholine | 0.75 mM |
| Sodium phosphate | 28.7 mM |
| Sodium chloride | 105.8 mM |

The pH was adjusted to 6.5.

A separate solution of calcium chloride was prepared by dissolving 149.2 mM of the salt in deionised water.

5.0 ml of FaSSIF was added to each of 7 glass vials. A known volume, varying from 0 to 0.5 ml, of the calcium chloride solution was added to each vial. Each sample was inspected visually immediately after the calcium chloride addition.

A volume of 1.0 ml was withdrawn from each sample and centrifuged for 20 mins at 14 000 rpm. The clear supernatant of each sample was collected and analysed with respect to bile acid content. The analyses were carried out using a bile acid analysis kit which employs an enzymatic colour reaction. The concentration of bile acid is proportional to the colour intensity which is determined by spectrophotometry.

Table A. The Effect of Calcium Chloride Addition to FaSSIF on the Taurocholate Concentration as Reflected in the Sample Absorbance after the Enzymatic Colour Reaction.

TABLE A

| | Added amount of | |
|---|---|---|
| Sample | calcium chloride (μmol) | Absorbance |
| A | 0 | 0.0943 |
| B | 7.5 | 0.0933 |
| C | 14.9 | 0.0890 |
| D | 22.4 | 0.0843 |
| E | 29.8 | 0.0783 |
| F | 44.8 | 0.0735 |
| G | 74.6 | 0.0718 |

A precipitate was formed in all samples immediately after calcium chloride was added. Furthermore, the amount of precipitation appeared to increase with increasing added volume of the calcium chloride solution. The bile acid analyses shows that the concentration of taurocholate in the aqueous solution decreased with increasing added amount of calcium chloride.

### Example B

### Reduction of the Concentration of Bile Acids in Aqueous Solution Caused by Addition of Calcium Chloride

A solution containing a mixture of bile acids was prepared by dissolving the following components in deionised water:

| | |
|---|---|
| Sodium lithocholate | 0.27 mM |
| Sodium deoxycholate | 2.2 mM |
| Sodium ursodeoxycholate | 0.34 mM |
| Sodium cholate | 0.24 mM |
| E-phosphatidylcholine | 0.74 mM |
| TES buffer | 30.3 mM |
| Sodium chloride | 100.1 mM |

The pH was adjusted to 7.4.

A calcium chloride solution was prepared by dissolving the following components in deionised water:

| | |
|---|---|
| Calcium chloride | 200.2 mM |
| TES buffer | 30.3 mM |
| Sodium chloride | 100.1 mM |

The pH was adjusted to 7.4.

2.0 ml of the bile acid solution was added to each of 6 glass vials. A known volume, varying from 0 to 300 μl, of the calcium chloride solution was added to each vial. Each sample was inspected visually immediately after the calcium chloride addition. 1.5 ml of each sample was transferred into a centrifugation tube and centrifuged for 20 mins at 14 000 rpm. The clear supernatant was collected and analysed with respect to bile acid content. The analyses were carried out using a bile acid analysis kit which employs an enzymatic colour reaction. The concentration of bile acid is proportional to the colour intensity which is determined by spectrophotometry.

Table B. The Effect of Addition of Calcium Chloride on the Bile Acid Concentration.

TABLE B

| | Added amount of | Concentration of |
|---|---|---|
| Sample | calcium chloride (μmol) | bile acids (mM) |
| A | 0 | 2.9 |
| B | 3.0 | 2.2 |
| C | 6.0 | 2.1 |
| D | 12.0 | 1.9 |
| E | 30.0 | 0.8 |
| F | 60.1 | 0.7 |

Again, a precipitate was formed in all samples immediately after calcium chloride was added. Furthermore, the amount of precipitation appeared to increase with increasing added amount of calcium chloride. The bile acid analyses shows that the concentration of bile acids in the aqueous solution decreased with increasing added amount of calcium chloride.

### Example C

### Reduction of the Concentration of Sodium Glycodeoxycholate (GDC) in Aqueous Solution Caused by Addition of Calcium Phosphate

A stock solution of sodium glycodeoxycholate (GDC) was prepared by dissolving the following substances in deionised water:

| | |
|---|---|
| Sodium glycodeoxycholate (GDC) | 15.0 mM |
| Sodium phosphate | 28.9 mM |
| Sodium chloride | 106 mM |

The pH was adjusted to 7.4 with sodium hydroxide.

A similar buffer solution with the same content, except for the bile acid was also prepared.

200 mg calcium phosphate (crystalline) was weighed into each of 10 glass vials labelled A-J. The GDC stock solution and the buffer solution were added in various proportions to the samples so that the total solution volume in each sample was 10 ml. The resulting initial GDC concentrations in the samples were 1-15 mM. The samples were equilibrated for several hours. The solid material in the samples were removed by centrifugation and/or filtration, and the obtained clear supernatants were analysed with respect to GDC content. The analyses were carried out by HPLC.

[image]

[image]

The results of the analyses show that the GDC concentration had been reduced by the presence of calcium phosphate in all samples.

### Example D

### Reduction of the Concentration of Sodium Deoxycholate (DC) in Aqueous Solution Caused by Addition of Calcium Phosphate

A stock solution of sodium deoxycholate (DC) was prepared by dissolving the following substances in deionised water:

| | |
|---|---|
| Sodium glycodeoxycholate (DC) | 20.1 mM |
| Sodium phosphate | 28.9 mM |
| Sodium chloride | 106 mM |

The pH was adjusted to 7.4 with sodium hydroxide.

A similar buffer solution with the same content, except for the bile acid was also prepared.

200 mg calcium phosphate (crystalline) was weighed into each of 9 glass vials labelled A-I. The DC stock solution and the buffer solution were added in various proportions to the samples so that the total solution volume in each sample was 10 ml. The resulting initial DC concentrations in the samples were 1-20 mM. The samples were equilibrated for several hours. The solid material in the samples were removed by centrifugation and/or filtration, and the obtained clear supernatants were analysed with respect to DC content. The analyses were carried out by HPLC.

[image]

The results of the analyses clearly showed that the DC concentration had been reduced by the presence of calcium phosphate in all samples.

Colon fistulated dogs may be used to demonstrate the effectiveness of the combination of the present invention in preventing diarrhoea. The IBAT inhibitor is dosed orally at a dose that will cause diarrhoea, for example 25-50 μmol/kg. The metal salt is then introduced into the colon, through the fistulae, to see if the diarrhoea can be prevented. The dose of the metal salt varies and can be determined after analysing the bile acid concentration in faeces from dogs having been exposed to the same dose of the IBAT inhibitor. The following example (Examples E) illustrates how to measure the lowering effect of a metal salt of the bile acid concentration in vivo.

### Example E

### In Vivo Reduction of the Bile Acid Concentration in the Feacal Aqueous Phase of the Dog Treated with an IBAT Inhibitor by Intracolonic Administration of Calcium Chloride

Labrador dogs with a colon fistula were used for studying the effect of intracolonic administration of an aqueous calcium chloride solution on the bile acid content in faecal water of dogs treated with an IBAT inhibitor.

A solution of an BAT inhibitor was administered directly into the stomach of the dog via an orogastric tube (t=0 hours). The dog was fed 30 minutes after the administration of the IBAT inhibitor (t=0.5 hours). The calcium chloride solution was administered 60 minutes after the IBAT inhibitor dosing (t=1 hour).

Faeces was collected during the first 8 hours after administration, and the time for each bowel movement was recorded. Each faeces sample was homogenized with a high-shear mixer and, subsequently, centrifuged in order to separate the solid material from the faecal water phase. The faecal water was collected and analysed with respect to bile acid content. The amount of bile acid in the faecal water was related to the amount of solid material in each faeces sample.

[image]

The results show that as long as calcium chloride is present in the colon, the bile acid concentration is relatively constant. After approximately 3.5 hours most of the calcium chloride has been removed from the colon, either by absorption or by the bowel movements. At this point, the IBAT inhibitor is still active at its site of action and the flow of bile acids into the colon is still substantial. The absence of calcium chloride in the colon allows for high bile acid concentration in the faecal output.

According to another feature of the invention there is provided the use of a metal salt, wherein the metal salt is formulated to release in the terminal ileum, caecum and/or the colon, for the prevention of diarrhoea that would result from excess bile acids in the intestine following administration of an IBAT inhibitor, or a pharmaceutically acceptable salt, solvate, solvate of such a salt or a prodrug thereof.

According to another feature of the invention there is provided the use of a metal salt, wherein the metal salt is formulated to release in the terminal ileum, caecum and/or the colon, in the manufacture of a medicament for the prevention of diarrhoea that would result from excess bile acids in the intestine following administration of an IBAT inhibitor, or a pharmaceutically acceptable salt, solvate, solvate of such a salt or a prodrug thereof.

A method of preventing diarrhoea that would result from excess bile acids in the intestine following administration of an IBAT inhibitor, or a pharmaceutically acceptable salt, solvate, solvate of such a salt or a prodrug thereof, which comprises administering to a patient in need thereof, a metal salt, wherein the metal salt is formulated to release in the terminal ileum, caecum and/or the colon.

According to another feature of the invention there is provided the use of an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, in the manufacture of a medicament for use in the production of an IBAT inhibitory effect in a warm-blooded animal, such as man.

Suitably the production of an IBAT inhibitory effect means the treatment of hyperlipidaemic conditions. Suitably the production of an IBAT inhibitory effect means the treatment of dyslipidemic conditions and disorders such as hyperlipidaemia, hypertrigliceridemia, hyperbetalipoproteinemia (high LDL), hyperprebetalipoproteinemia (high VLDL), hyperchylomicronemia, hypolipoproteinemia, hypercholesterolemia, hyperlipoproteinemia and hypoalphalipoproteinemia (low HDL). Suitably the production of an IBAT inhibitory effect means the treatment of different clinical conditions such as atherosclerosis, arteriosclerosis, arrhythmia, hyper-thrombotic conditions, vascular dysfunction, endothelial dysfunction, heart failure, coronary heart diseases, cardiovascular diseases, myocardial infarction, angina pectoris, peripheral vascular diseases, inflammation of cardiovascular tissues such as heart, valves, vasculature, arteries and veins, aneurisms, stenosis, restenosis, vascular plaques, vascular fatty streaks, leukocytes, monocytes and/or macrophage infiltration, intimal thickening, medial thinning, infectious and surgical trauma and vascular thrombosis, stroke and transient ischaemic attacks. Suitably the production of an IBAT inhibitory effect means the treatment of atherosclerosis, coronary heart diseases, myocardial infarction, angina pectoris, peripheral vascular diseases, stroke and transient ischaemic attacks.

According to another feature of the invention there is provided the use of a metal salt, wherein the metal salt is formulated to release in the terminal ileum, caecum and/or the colon, in the manufacture of a medicament for use in preventing diarrhoea that would result from excess bile acids in the intestine following administration of an IBAT inhibitor, or a pharmaceutically acceptable salt, solvate, solvate of such a salt or a prodrug thereof, which medicament comprises an IBAT inhibitor, or a pharmaceutically acceptable salt, solvate, solvate of such a salt or a prodrug thereof in combination with a metal salt, wherein the metal salt is formulated to release in the terminal ileum, caecum and/or the colon.

According to a further feature of this aspect of the invention there is provided a method for producing an IBAT inhibitory effect in a warm-blooded animal, such as man, in need of such treatment which comprises administering to said animal an effective amount of an IBAT inhibitor, or a pharmaceutically acceptable salt, solvate, solvate of such a salt or a prodrug thereof in combination with a metal salt, wherein the metal salt is formulated to release in the terminal ileum, caecum and/or the colon.

Therefore according to the present invention, there is provided a method of preventing diarrhoea that would result from excess bile acids in the intestine following administration of an effective amount an IBAT inhibitor, or a pharmaceutically acceptable salt, solvate, solvate of such a salt or a prodrug thereof, in a warm blooded animal, such as man, in need of such treatment, which comprises administering to said animal said effective amount of an IBAT inhibitor, or a pharmaceutically acceptable salt, solvate, solvate of such a salt or a prodrug thereof in combination with a metal salt, wherein the metal salt is formulated to release in the terminal ileum, caecum and/or the colon.

According to a further aspect of the invention there is provided a pharmaceutical composition which comprises an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, in association with a pharmaceutically acceptable diluent or carrier.

According to a further aspect of the invention there is provided a pharmaceutical composition which comprises an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, in association with a pharmaceutically acceptable diluent or carrier for use in producing an IBAT inhibitory effect, in a warm-blooded animal, such as man.

According to a further aspect of the invention there is provided a pharmaceutical composition which comprises an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, in association with a pharmaceutically acceptable diluent or carrier; for use in preventing diarrhoea that would result from excess bile acids in the intestine following administration of an IBAT inhibitor, or a pharmaceutically acceptable salt, solvate, solvate of such a salt or a prodrug thereof, in a warm-blooded animal, such as man.

The pharmaceutical compositions may be in a form suitable for oral administration, for example as a tablet or capsule. In general the above compositions may be prepared in a conventional manner using conventional excipients.

According to an additional feature of the invention, there is provided an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, for use as a medicament.

According to an additional feature of the invention, there is provided an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, for use in producing an IBAT inhibitory effect, in a warm-blooded animal, such as man.

According to an additional feature of the invention, there is provided an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, for use in preventing diarrhoea that would result from excess bile acids in the intestine following administration of an IBAT inhibitor, or a pharmaceutically acceptable salt, solvate, solvate of such a salt or a prodrug thereof, to a warm-blooded animal, such as man.

According to a further aspect of the present invention there is provided a kit comprising an IBAT inhibitor, or a pharmaceutically acceptable salt, solvate, solvate of such a salt or a prodrug thereof, and a metal salt, wherein the metal salt is formulated to release in the terminal ileum, caecum and/or the colon, optionally with instructions for use.

According to a further aspect of the present invention there is provided a kit comprising an IBAT inhibitor, or a pharmaceutically acceptable salt, solvate, solvate of such a salt or a prodrug thereof, and a metal salt, wherein the metal salt is formulated to release in the terminal ileum, caecum and/or the colon, optionally with instructions for use; for use in producing an IBAT inhibitory effect, in a warm-blooded animal, such as man.

According to a further aspect of the present invention there is provided a kit comprising an IBAT inhibitor, or a pharmaceutically acceptable salt, solvate, solvate of such a salt or a prodrug thereof, and a metal salt, wherein the metal salt is formulated to release in the terminal ileum, caecum and/or the colon; optionally with instructions for use; for use in preventing diarrhoea that would result from excess bile acids in the intestine following administration of an IBAT inhibitor, or a pharmaceutically acceptable salt, solvate, solvate of such a salt or a prodrug thereof, to a warm-blooded animal, such as man.

According to a further aspect of the present invention there is provided a kit comprising:

a) an IBAT inhibitor, or a pharmaceutically acceptable salt, solvate, solvate of such a salt or a prodrug thereof, in a first unit dosage form;

b) a metal salt, wherein the metal salt is formulated to release in the terminal ileum, caecum and/or the colon; in a second unit dosage form; and

c) container means for containing said first and second dosage forms; and optionally

d) with instructions for use.

According to a further aspect of the present invention there is provided a kit comprising:

a) an IBAT inhibitor, or a pharmaceutically acceptable salt, solvate, solvate of such a salt or a prodrug thereof, in a first unit dosage form;

b) a metal salt, wherein the metal salt is formulated to release in the terminal ileum, caecum and/or the colon; in a second unit dosage form; and

c) container means for containing said first and second dosage forms; and optionally

d) with instructions for use;

for use in producing an IBAT inhibitory effect, in a warm-blooded animal, such as man.

According to a further aspect of the present invention there is provided a kit comprising:

a) an IBAT inhibitor, or a pharmaceutically acceptable salt, solvate, solvate of such a salt or a prodrug thereof, in a first unit dosage form;

b) a metal salt, wherein the metal salt is formulated to release in the terminal ileum, caecum and/or the colon; in a second unit dosage form; and

c) container means for containing said first and second dosage forms; and optionally

d) with instructions for use;

for use in preventing diarrhoea that would result from excess bile acids in the intestine following administration of an IBAT inhibitor, or a pharmaceutically acceptable salt, solvate, solvate of such a salt or a prodrug thereof, to a warm-blooded animal, such as man.

According to a further aspect of the present invention there is provided a combination comprising an IBAT inhibitor, or a pharmaceutically acceptable salt, solvate, solvate of such a salt or a prodrug thereof, and a metal salt, wherein the metal salt is formulated to release in the terminal ileum, caecum and/or the colon, for use in producing an IBAT inhibitory effect, in a warm-blooded animal, such as man.

According to a further aspect of the present invention there is provided a combination comprising an IBAT inhibitor, or a pharmaceutically acceptable salt, solvate, solvate of such a salt or a prodrug thereof, and a metal salt, wherein the metal salt is formulated to release in the terminal ileum, caecum and/or the colon, for use in preventing diarrhoea that would result from excess bile acids in the intestine following administration of an IBAT inhibitor, or a pharmaceutically acceptable salt, solvate, solvate of such a salt or a prodrug thereof, to a warm-blooded animal, such as man.

According to a further aspect of the present invention there is provided a combination treatment comprising the administration of an effective amount of an IBAT inhibitor, or a pharmaceutically acceptable salt, solvate, solvate of such a salt or a prodrug thereof, optionally together with a pharmaceutically acceptable diluent or carrier, in combination with an effective amount of a metal salt, wherein the metal salt is formulated to release in the terminal ileum, caecum and/or the colon, optionally together with a pharmaceutically acceptable diluent or carrier; to a warm-blooded animal, such as man in need of such therapeutic treatment.

According to a further aspect of the present invention there is provided a combination treatment comprising the administration of an effective amount of an IBAT inhibitor, or a pharmaceutically acceptable salt, solvate, solvate of such a salt or a prodrug thereof, optionally together with a pharmaceutically acceptable diluent or carrier, in combination with an effective amount of a metal salt, wherein the metal salt is formulated to release in the terminal ileum, caecum and/or the colon, optionally together with a pharmaceutically acceptable diluent or carrier for use in producing an IBAT inhibitory effect, in a warm-blooded animal, such as man.

According to a further aspect of the present invention there is provided a combination treatment comprising the administration of an effective amount of an IBAT inhibitor, or a pharmaceutically acceptable salt, solvate, solvate of such a salt or a prodrug thereof, optionally together with a pharmaceutically acceptable diluent or carrier, in combination with an effective amount of a metal salt, wherein the metal salt is formulated to release in the terminal ileum, caecum and/or the colon, optionally together with a pharmaceutically acceptable diluent or carrier; for use in preventing diarrhoea that would result from excess bile acids in the intestine following administration of an IBAT inhibitor, or a pharmaceutically acceptable salt, solvate, solvate of such a salt or a prodrug thereof, to a warm-blooded animal, such as man.

The IBAT inhibitor, or a pharmaceutically acceptable salt, solvate, solvate of such a salt or a prodrug thereof, will normally be administered to a warm-blooded animal at a unit dose within the range 5-5000 mg per square meter body area of the animal, i.e. approximately 0.01-50 mg/kg, and this would be expected to provide a therapeutically-effective dose. A unit dose from such as a tablet or capsule will usually contain, for example 1-250 mg of active ingredient. In one aspect of the invention a daily dose in the range of 0.02-50 mg/kg is employed. In another aspect a daily dose in the rage of 0.02-20 mg/kg is employed. In another aspect of the invention the compound of formula (1), or a pharmaceutically acceptable salt, solvate, solvate of such a salt or a prodrug thereof, will normally be administered to a warm-blooded animal at a unit dose within the range 0.001-20 mg/kg or 0.1-200 mg/day, particularly 1-20 mg/day to provide a therapeutically-effective dose. However the daily dose will necessarily be varied depending upon the host treated, the particular route of administration, and the severity of the illness being treated. Accordingly the optimum dosage may be determined by the practitioner who is treating any particular patient.

The metal salt will normally be administered to a warm-blooded animal at a unit dose which will be varied depending upon the host treated, the particular route of administration, and the severity of the illness being treated. Accordingly the optimum dosage may be determined by the practitioner who is treating any particular patient. Suitably this dose will be 2 g or less per patient per day. Suitably this dose will be 1 g or less per patient per day. More suitably it will be 500 mg or less per patient per day. In another aspect a daily dose in the range of 50-100 mg per day is employed.

The dosage of each of the two drugs and their proportions have to be composed so that the best possible treatment effects, as defined by national and international guidelines (which are periodically reviewed and re-defined), will be met.

For the avoidance of doubt, where the prevention of diarrhoea that would result from excess bile acids in the intestine following administration of an IBAT inhibitor, or a pharmaceutically acceptable salt, solvate, solvate of such a salt or a prodrug thereof is referred to, it is to be understood that this also refers to the treatment of diarrhoea that has resulted from excess bile acids in the intestine following administration of an IBAT inhibitor, or a pharmaceutically acceptable salt, solvate, solvate of such a salt or a prodrug thereof.

The combination therapy defined hereinbefore may also involve, in addition to the combination, one or more other substances and/or treatments. Such conjoint treatment may be achieved by way of the simultaneous, sequential or separate administration of the individual components of the treatment.

Suitable additional substances include MG Co-A reductase inhibitors, or pharmaceutically acceptable salts, solvates, solvates of such salts or prodrugs thereof. Suitable HMG Co-A reductase inhibitors, pharmaceutically acceptable salts, solvates, solvates of such salts or prodrugs thereof are statins well known in the art. Particular statins are fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof. A particular statin is atorvastatin, or a pharmaceutically acceptable salt, solvate, solvate of such a salt or a prodrug thereof. A more particular statin is atorvastatin calcium salt. A further particular statin is rosuvastatin, or a pharmaceutically acceptable salt, solvate, solvate of such a salt or a prodrug thereof. A preferable particular statin is rosuvastatin calcium salt.

Further suitable additional substances include:
a CETP (cholesteryl ester transfer protein) inhibitor, for example those referenced and described in WO 00/38725 page 7 line 22—page 10, line 17 which are incorporated herein by reference;
a cholesterol absorption antagonist for example azetidinones such as SCH 58235 and those described in U.S. Pat. No. 5,767,115 which are incorporated herein by reference;
a MTP (microsomal transfer protein) inhibitor for example those described in Science, 282, 751-54, 1998 which are incorporated herein by reference;
a fibric acid derivative; for example clofibrate, gemfibrozil, fenofibrate, ciprofibrate and bezafibrate;
a nicotinic acid derivative, for example, nicotinic acid (niacin), acipimox and niceritrol;
a phytosterol compound for example stanols;
probucol;
an anti-obesity compound for example orlistat (EP 129,748) and sibutramine (GB 2,184,122 and U.S. Pat. No. 4,929,629);
an antihypertensive compound for example an angiotensin converting enzyme (ACE) inhibitor, an angiotensin II receptor antagonist, an andrenergic blocker, an alpha andrenergic blocker, a beta andrenergic blocker, a mixed alpha/beta andrenergic blocker, an andrenergic stimulant, calcium channel blocker, a diuretic or a vasodilator;
insulin;
sulphonylureas including glibenclamide, tolbutamide;
metformin; and/or
acarbose;
   or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Particular ACE inhibitors or pharmaceutically acceptable salts, solvates, solvate of such salts or a prodrugs thereof, including active metabolites, which can be used as an additional substance include but are not limited to, the following compounds: alacepril, alatriopril, altiopril calcium, ancovenin, benazepril, benazepril hydrochloride, benazeprilat, benzoylcaptopril, captopril, captopril-cysteine, captopril-glutathione, ceranapril, ceranopril, ceronapril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, glycopril, hemorphin-4, idrapril, imidapril, indolapril, indolaprilat, libenzapril, lisinopril, lyciumin A, lyciumin B, mixanpril, moexipril, moexiprilat, moveltipril, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, utibapril, zabicipril, zabiciprilat, zofenopril and zofenoprilat. Preferred ACE inhibitors for use in the present invention are ramipril, ramiprilat, lisinopril, enalapril and enalaprilat. More preferred ACE inhibitors for uses in the present invention are ramipril and ramiprilat.

Preferred angiotensin II antagonists, pharmaceutically acceptable salts, solvates, solvate of such salts or a prodrugs thereof for use as an additional substance, include but are not limited to candesartan, candesartan cilexetil, losartan, valsartan, irbesartan, tasosartan, telmisartan and eprosartan. Particularly preferred angiotensin U antagonists or pharmaceutically acceptable derivatives thereof for use in the present invention are candesartan and candesartan cilexetil.

Additional suitable additional substances are PPAR alpha and/or gamma agonists, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma agonist refers to WY-14643, clofibrate, fenofibrate, bezafibrate, GW 9578, troglitazone, pioglitazone, rosiglitazone, eglitazone, proglitazone, BRL-49634, KRP-297, JIT-501, SB 213068, GW 1929, GW 7845, GW 0207, L-796449, L-165041 and GW 2433. Particularly a PPAR alpha and/or gamma agonist refers to (S)-2-ethoxy-3-[4-(2-{4-methanesulphonyloxyphenyl}ethoxy)phenyl] propanoic acid and pharmaceutically acceptable salts thereof.

Therefore in a further aspect of the invention there is provided a combination which comprises an IBAT inhibitor, or a pharmaceutically acceptable salt, solvate, solvate of such a salt or a prodrug thereof, and a metal salt, wherein the metal salt is formulated to release in the terminal ileum, caecum and/or the colon, and one or more suitable additional substances as defined herein above.

According to another feature of the invention there is provided the use of an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, and one or more suitable additional substances as defined herein above in the production of an BAT inhibitory effect in a warm-blooded animal, such as man.

According to another feature of the invention there is provided the use of an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, and one or more suitable additional substances as defined herein above, in the manufacture of a medicament for use in the production of an IBAT inhibitory effect in a warm-blooded animal, such as man.

According to a further feature of this aspect of the invention there is provided a method for producing an IBAT inhibitory effect in a warm-blooded animal, such as man, in need of such treatment which comprises administering to said animal an effective amount of an IBAT inhibitor, or a pharmaceutically acceptable salt, solvate, solvate of such a salt or a prodrug thereof in combination with a metal salt, wherein the metal salt is formulated to release in the terminal ileum, caecum and/or the colon, and one or more suitable additional substances as defined herein above.

According to a further aspect of the invention there is provided a pharmaceutical composition which comprises an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, and one or more suitable additional substances as defined herein above, in association with a pharmaceutically acceptable diluent or carrier.

According to a further aspect of the invention there is provided a pharmaceutical composition which comprises an IBAT inhibitor, or a pharmaceutically acceptable salt, solvate, solvate of such a salt or a prodrug thereof, in combination with a metal salt, wherein the metal salt is formulated to release in the terminal ileum, caecum and/or the colon, and one or more suitable additional substances as defined herein above, in association with a pharmaceutically acceptable diluent or carrier for use in producing an IBAT inhibitory effect, in a warm-blooded animal, such as man.

The metal salt can be formulated in a delayed release single or multiple unit oral formulation. The delayed release of the metal salt can be achieved by for example using techniques producing formulations with time dependent or pH dependent release or enzymatically degradable formulations (Pharmaceutics. The Science of Dosage Form Design Second Edition; Ed. Micheal E Aulton; Harcourt Publishers Limited; 2002). These formulations can be manufactured with conventional techniques, for example as described in Aulton, (see above), or Industrial Aspects of Pharmaceutics, Ed Erik Sandell; Swedish Pharmaceutical Press; 1993). Another reference illustrating how substances can be formulated to release in the colon is “Colonic Drug Delivery”, Watts et al, Drug Development and Industrial Pharmacy, 23(9), 893-913 (1997).

The IBAT inhibitor may be formulated by conventional techniques.

1. A combination which comprises an IBAT inhibitor, or a pharmaceutically acceptable salt, solvate, solvate of such a salt or a prodrug thereof, and a metal salt, wherein the metal salt is formulated to release in the terminal ileum, caecum and/or the colon.

2. A combination according to claim 1 wherein the metal salt is a calcium salt.

3. A combination according to claim 1 wherein the metal salt is calcium phosphate.

4. A combination according to claim 1 wherein the IBAT inhibitor is a benzothiepine.

5. A combination according to claim 1 wherein the IBAT inhibitor is selected from:
1,1-dioxo-3,37-dibutyl-5-phenyl-7-methylthio-8-(N-{(R)-1′-phenyl-1′-[N′-(carboxymethyl) carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(carboxymethyl)carbamoyl]-4-hydroxybenzyl}carbamoylmethoxy)-2,3,4,5-tetrahydro-1,5-benzothiazepine;
1,1-dioxo-3,3-dibutyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3-butyl-3-ethyl-5-phenyl-7-methylthio-8-(N-{(R)-1′-phenyl-1′-[N′-(2-sulphoethyl)carbamoyl]methyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-sulphoethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-sulphoethyl) carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]-4-hydroxybenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(5-carboxypentyl) carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(2-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{α-[N′-(2-sulphoethyl)carbamoyl]-2-fluorobenzyl}carbamoylmethoxy)-2,3,4,5-tetrahydro-1,5-benzothiazepine;
1,1-dioxo-3-butyl-3-ethyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-(N-{(R)-α-[N′-(R)-(2-hydroxy-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{(R)-1-[N″-(R)-(2-hydroxy-1-carboxyethyl)carbamoyl]-2-hydroxyethyl}carbamoyl)benzyl]carbamoylmethoxy}-2,3,4,5-tetrahydro-1,5-benzothiazepine;
1,1-dioxo-3-butyl-3-ethyl-5-phenyl-7-methylthio-8-(N-{α-[N′-(carboxymethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-(N-{α-[N′-((ethoxy)(methyl)phosphorylmethyl)carbamoyl]benzyl}carbamoylmethoxy)-2,3,4,5-tetrahydro-1,5-benzothiazepine;
1,1-dioxo-3-butyl-3-ethyl-5-phenyl-7-methylthio-8-{N-[(R)-α-(N′-{2-[(hydroxy)(methyl)phosphoryl]ethyl}carbamoyl)benzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[N′-(2-methylthio-1-carboxyethyl)carbamoyl]benzyl}carbamoylmethoxy)-2,3,4,5-tetrahydro-1,5-benzothiazepine;
1,1-dioxo-3,3-dibutyl-5-phenyl-7-methylthio-8-{N-[(R)-α-(N′-{2-[(methyl)(ethyl) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-{N-[(R)-α-(N′-{2-[(methyl)(hydroxy) phosphoryl]ethyl}carbamoyl)-4-hydroxybenzyl]carbamoylmethoxy}-2,3,4,5-tetrahydro-1,5-benzothiazepine;
1,1-dioxo-3,3-dibutyl-5-phenyl-7-methylthio-8-(N-{(R)-α-[(R)-N′-(2-methylsulphinyl-1-carboxyethyl)carbamoyl]benzyl}carbamoylmethoxy)-2,3,4,5-tetrahydro-1,5-benzothiazepine; and
1,1-dioxo-3,3-dibutyl-5-phenyl-7-methoxy-8-[N-{(R)-α-[N′-(2-sulphoethyl)carbamoyl]-4-hydroxybenzyl}carbamoylmethoxy]-2,3,4,5-tetrahydro-1,5-benzothiazepine;
or a pharmaceutically acceptable salt, solvate, solvate of such a salt or a prodrug thereof.

6. A combination according to claim 1 any on of claims wherein the IBAT inhibitor is selected from:
1,1-dioxo-3,3-dibutyl-5-phenyl-7-methylthio-8-(N-{(R)-α-[N-((R)-1-carboxy-2-methylthioethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxybutyl) carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl) carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-(R)-hydroxypropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxyethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((R)-1-carboxy-2-methylthioethyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-{(S)-1-[N-((S)-2-hydroxy-1-carboxyethyl)carbamoyl]propyl}carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxy-2-methylpropyl)carbamoyl]benzyl}carbamoylmethoxy)-2,3,4,5-tetrahydro-1,2,5-benzothiadiazepine;
1,1-dioxo-3,3-dibutyl-5-phenyl-7-methylthio-8-(N-{(R)-α-[N-((S)-1-carboxypropyl) carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-[N-((R)-α-carboxy-4-hydroxybenzyl)carbamoylmethoxy]-2,3,4,5-tetrahydro-1,2,5-benzothiadiazepine;
or a pharmaceutically acceptable salt, solvate, solvate of such a salt or a prodrug thereof.

7-8. (canceled)

9. A method for producing an IBAT inhibitory effect in a warm-blooded animal, such as man, in need of such treatment which comprises administering to said animal an effective amount of a combination according to claim 1.

10. A method of preventing diarrhoea that would result from excess bile acids in the intestine following administration of an effective amount an IBAT inhibitor, or a pharmaceutically acceptable salt, solvate, solvate of such a salt or a prodrug thereof, to a warm blooded animal, such as man, in need of such treatment, which comprises administering to said animal an effective amount of a combination according to claim 1.

11. A pharmaceutical composition which comprises a combination according to claim 1, in association with a pharmaceutically acceptable diluent or carrier.

12-14. (canceled)

15. A method of treating hyperlipidaemic conditions in a warm-blooded animal, such as man, in need of such treatment which comprises administering to said animal an effective amount of a combination according to claim 1.

16-18. (canceled)

19. The combination according to claim 1 further comprising an HMG Co-A reductase inhibitor, or pharmaceutically acceptable salts, solvates, solvates of such salts or prodrugs thereof.

20. The combination according to claim 19 wherein the HMG Co-A reductase inhibitor is fluvastatin, lovastatin, pravastatin, simvastatin, atorvastatin, cerivastatin, bervastatin, dalvastatin, mevastatin and rosuvastatin, or a pharmaceutically acceptable salt, solvate, solvate of such a salt or a prodrug thereof.

21. The combination according to claim 1 further comprising a cholesterol absorption antagonist, or pharmaceutically acceptable salts, solvates, solvates of such salts or prodrugs thereof.

22. The combination according to claim 21 wherein the a cholesterol absorption antagonist is SCH 58235.

23. The combination according to claim 1 further comprising a PPAR alpha and/or gamma agonist, or pharmaceutically acceptable salts, solvates, solvates of such salts or prodrugs thereof.

24. The combination according to claim 23 wherein the PPAR alpha and/or gamma agonist is (S)-2-ethoxy-3-[4-(2-{4-methanesulphonyloxyphenyl}ethoxy)phenyl]propanoic acid and pharmaceutically acceptable salts thereof.

25-26. (canceled)

27. A method for producing an IBAT inhibitory effect in a warm-blooded animal, such as man, in need of such treatment which comprises administering to said animal an effective amount of a composition according to claim 19.

28. A pharmaceutical composition which comprises a combination according to claim 19, in association with a pharmaceutically acceptable diluent or carrier.

29-31. (canceled)

32. A method of preventing diarrhoea that would result from excess bile acids in the intestine following administration of an IBAT inhibitor, or a pharmaceutically acceptable salt, solvate, solvate of such a salt or a prodrug thereof, which comprises administering to a patient in need thereof, a metal salt, wherein the metal salt is formulated to release in the terminal ileum, caecum and/or the colon.
